(19) United States
(12) Patent Application Publication
(10) Pub. No.: US 2026/0334812 A1
(43) Pub. Date: Oct. 29, 2026

(54) COMBINED TESTING AND TREATMENT UNIT WITH CONSUMABLE CARTRIDGE SYSTEM

(71) Applicants: KATHRYN CASHMAN ROGERS, DALLAS, TX (US); CHRIS ROGERS, DALLAS, TX (US)

(72) Inventors: KATHRYN CASHMAN ROGERS, DALLAS, TX (US); CHRIS ROGERS, DALLAS, TX (US)

(21) Appl. No.: 19/726,612

(22) Filed: Jun. 30, 2026

Related U.S. Application Data
(63) Continuation-in-part of application No. 17/985,947, filed on Nov. 14, 2022, now Pat. No. 12,667,305.
(60) Provisional application No. 63/278,322, filed on Nov. 11, 2021.
(60) Provisional application No. 64/039,832, filed on Apr. 15, 2026.

(51) Int. Cl. A61B 5/097; A61B 5/00; A61B 5/08; A61B 5/087; A61M 15/00

(52) U.S. Cl. CPC A61B 5/097; A61B 5/0002; A61B 5/082; A61B 5/087; A61M 15/0021; A61M 15/0085; A61M 2205/123

(57) ABSTRACT

A combined or convertible handheld unit that combines a personal spirometer, exhalation-analysis or breath-analysis function with a nebulizer or vaporizing function. The nebulizer or vaporizing function includes, but is not limited to, functions provided by devices such as inhalers, nebulizers, vaporizers, humidifiers, ventilators, or other devices that are used for delivery of any sort of substance into the body via breathing, and includes but is not limited to devices that break up solutions and suspensions into small aerosol droplets for inhalation by a user. The unit also performs full exhalation detection and analytics.

This application is a continuation-in-part application of U.S. patent application Ser. No. 17/985,947, filed Nov. 14, 2022, published as US 2023/0277124 on Sep. 7, 2023, now issued as U.S. Pat. No. 12,667,305 on Jun. 30, 2026, which claims benefit of and priority to U.S. Provisional App. No. 63/278,322, filed Nov. 11, 2021. The application also claims benefit of and priority to U.S. Provisional App. No. 64/039,832, filed Apr. 15, 2026. All of the above references are incorporated herein in their entireties by specific reference for all purposes.

### FIELD OF INVENTION

The present invention relates to a system for managing health and related information in connection with use of a personal testing and treatment device or unit. More particularly, this invention relates to a computer-based system and applications for managing health and related information through use of a handheld personal device that generates or nebulizes a vapor for inhalation by a user using consumable cartridges, and that provides exhalation or breath analysis.

### BACKGROUND OF INVENTION

Various devices for inhalation, nebulizing or vaporizing functions are known in the prior art, as are devices for breath analysis or measurement of lung function. Examples include those described in Gueret, U.S. Pat. No. 5,529,055 (Jun. 25, 1996); Haber, et al., U.S. Pat. No. 5,435,282 (Jul. 25, 1995); van der Linden, et al., U.S. Pat. No. 5,950,619; Smith, et al., U.S. 2003/0140921 (Jul. 13, 2003); Star, et al., US 2010/0282245 (Nov. 11, 2010); Patton, et al., US 2013/0269694 (Oct. 17, 2013); Kotnik, et al., US 2008/0110452 (May 15, 2008); Albert, et al, US 2015/0014433 (Jan. 15, 2015); Arkush, US 2015/0258370 (Sep. 17, 2015); U.S. patent application Ser. No. 15/699,786 (filed Sep. 8, 2017); and Sakurada, et al., US 2010/0089394 (Apr. 15, 2010); all of which are incorporated herein in their entireties by specific reference for all purposes.

Sakurada discloses an inhaler capable of measuring lung function, but the lung function measurement uses the same components and fluid passageway as the inhaler, thereby allowing cross-contamination and interference between the two functions.

Accordingly, there exists a need for a modular device system providing both inhalation and exhalation functions that addresses the limitations of the prior art.

### SUMMARY OF INVENTION

In various exemplary embodiments, the present invention comprises a combined or convertible handheld modular unit that combines a personal spirometer, exhalation-analysis or breath-analysis function with a nebulizer or vaporizing function. The nebulizer or vaporizing function includes, but is not limited to, functions provided by devices such as inhalers, nebulizers, vaporizers, humidifiers, ventilators, or other devices that are used for delivery of any sort of substance into the body via breathing, and includes but is not limited to devices that break up solutions and suspensions into small aerosol droplets for inhalation by a user. In several embodiments, the unit also performs full exhalation detection and analytics.

Use and management of the unit may be coordinated with a computer-based program or application, which can run on a mobile computing device or other computing device. In various embodiments, the present invention comprises and uses the unit as a part of a computer-based system for receiving, analyzing, processing, managing and sending personal health information, and other information in conjunction with use of one or more personal nebulizer or vaporizing devices and personal breath analysis, exhalation analysis, or spirometer devices. In several embodiments, the system comprises a health or wellness computer application, program or database on a computer or mobile device (e.g., smart phone, tablet computer), which can coordinate collecting, storing, analyzing, and diagnosing various information obtained from a variety of other devices or sources, such as a breath analysis device that can capture and analyze the content of exhaled air, a health band (e.g., Fit Bit), other detection devices, or information entered into and stored in a separate health or wellness computer application, program or database on a computing device or mobile computing device. The system can use this information to develop health-related recommendations for an individual, including, but not limited to, recommendations for a substance or substances to be used in a personal vaporization device. Further, a particular breath analysis, when completed, the system real-time stamps the location (using GPS of the device) to gather a variety of relevant information about the location at that time, including, but not limited to, environmental factors, allergens present, weather, elevation, humidity, air quality index, and other similar factors. This information is then used to properly dose the user/patent for the current or next round of treatment.

In further embodiments, the system is modular, with particular components and functions in associated modules, where components such as a consumable cartridge module may be removably connected with one or more other reusable modules.

### BRIEF DESCRIPTION OF THE DRAWINGS

FIG. 1A shows a front perspective view of a device in accordance with an exemplary embodiment of the present invention.

FIG. 1B shows a side view of the device of FIG. 1A.

FIG. 2A shows a back view of the device of FIG. 1A.

FIG. 2B shows a back perspective view of the device of FIG. 1A.

FIG. 3A shows a front view of the device of FIG. 1A.

FIG. 3B shows a top view of the device of FIG. 1A.

FIG. 3C shows a bottom view of the device of FIG. 1A.

FIG. 4 shows an exploded perspective view of the device of FIG. 1A.

FIG. 5 shows a partial exploded perspective view of internal components of the device of FIG. 1A.

FIG. 6A shows a partial side view of assembled internal components of the device of FIG. 1A.

FIG. 6B shows a back view of the assembled internal components of FIG. 6A.

FIG. 6C shows a front view of the assembled internal components of FIG. 6B.

FIG. 7 shows an exploded perspective view of a two-tier modular system showing the cartridge with integrated piezoelectric transducer and the base device or unit.

FIG. 8 shows a cross-sectional view of the two-tier cartridge showing the liquid reservoir, piezoelectric transducer, and mouthpiece assembly with vapor/liquid separation mechanisms.

FIG. 9 shows a detailed view of the electrical contact interface between the cartridge and base device.

FIG. 10 shows an exploded perspective view of the three-tier system showing the cartridge, piezoelectric module, and base device.

FIG. 11 shows a cross-sectional view of the three-tier cartridge showing the liquid reservoir and sealed bottom membrane.

FIG. 12 shows a cross-sectional view of the piezoelectric module showing the seal-breaking mechanism and transducer.

FIG. 13 shows a detailed view of the module-cartridge interface showing seal-breaking mechanism engaging cartridge membrane.

FIG. 14 shows an exploded perspective view of a two-tier system with combined inhalation and exhalation functions.

FIG. 15 shows an exploded perspective view of a three-tier system with combined inhalation and exhalation functions.

FIG. 16 shows a side view of the system of FIG. 15.

FIG. 17 shows an exploded perspective view of an alternative embodiment of a two-tier system with combined inhalation and exhalation functions.

FIG. 18 shows an exploded perspective view of an alternative embodiment of a three-tier system with combined inhalation and exhalation functions.

### DETAILED DESCRIPTION OF EXEMPLARY EMBODIMENTS

In various exemplary embodiments, the present invention comprises a combined or convertible handheld unit 2 that combines a personal spirometer, exhalation-analysis or breath-analysis function with a nebulizer or vaporizing function. The nebulizer or vaporizing function includes, but is not limited to, functions provided by devices such as inhalers, nebulizers, vaporizers, humidifiers, ventilators, or other devices that are used for delivery of any sort of substance into the body via breathing, and includes but is not limited to devices that break up solutions and suspensions into small aerosol droplets for inhalation by a user. In several embodiments, the unit also performs full exhalation detection and analytics.

Use and management of the unit may be coordinated with a computer-based program or application, which can run on a mobile computing device or other computing device. In various embodiments, the present invention comprises and uses the unit as apart of a computer-based system for receiving, analyzing, processing, managing and sending personal health information, and other information in conjunction with use of one or more personal nebulizer or vaporizing devices and personal breath analysis, exhalation analysis, or spirometer devices. In several embodiments, the system comprises a health or wellness computer application, program or database on a computer or mobile device (e.g., smart phone, tablet computer), which can coordinate collecting, storing, analyzing, and diagnosing various information obtained from a variety of other devices or sources, such as a breath analysis device that can capture and analyze the content of exhaled air, a health band (e.g., Fit Bit), other detection devices, or information entered into and stored in a separate health or wellness computer application, program or database on a computing device or mobile computing device. The system can use this information to develop health-related recommendations for an individual, including, but not limited to, recommendations for a substance or substances to be used in a personal vaporization device. Further, a particular breath analysis, when completed, the system real-time stamps the location (using GPS of the device) to gather a variety of relevant information about the location at that time, including, but not limited to, environmental factors, allergens present, weather, elevation, humidity, air quality index, and other similar factors. This information is then used to properly dose the user/patent for the current or next round of treatment.

The system may be modular, with components contained in two or more separate modules that may be removably connected. In alternative embodiments, the system may be a single main unit or housing.

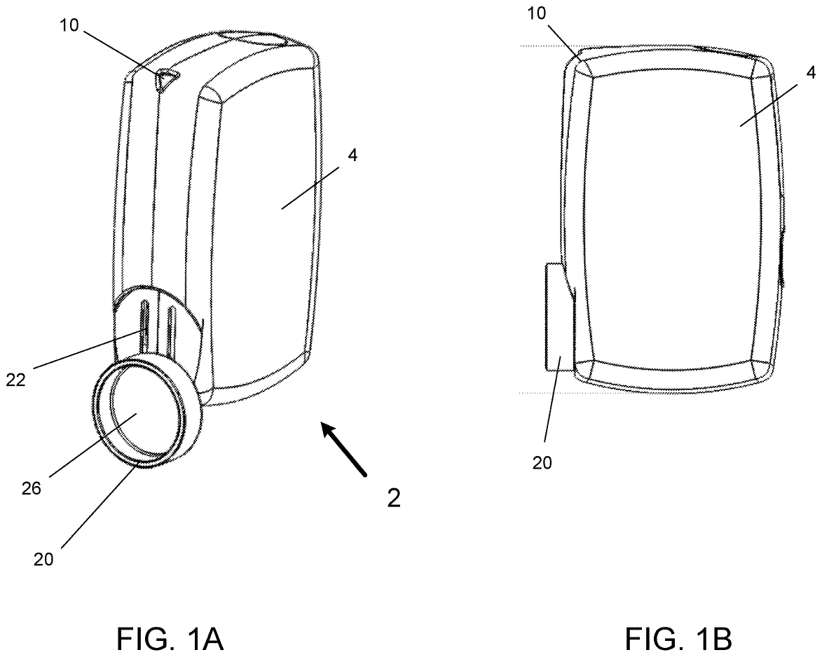

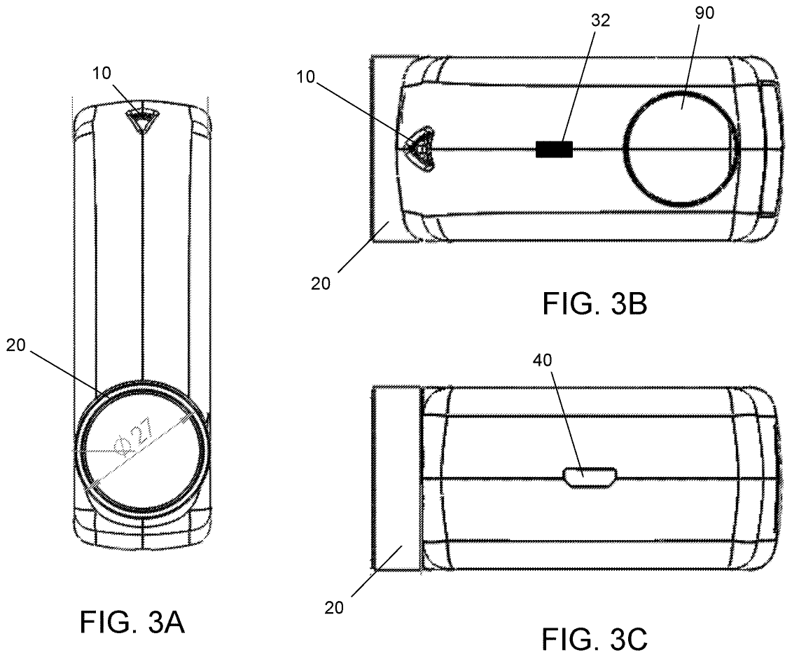

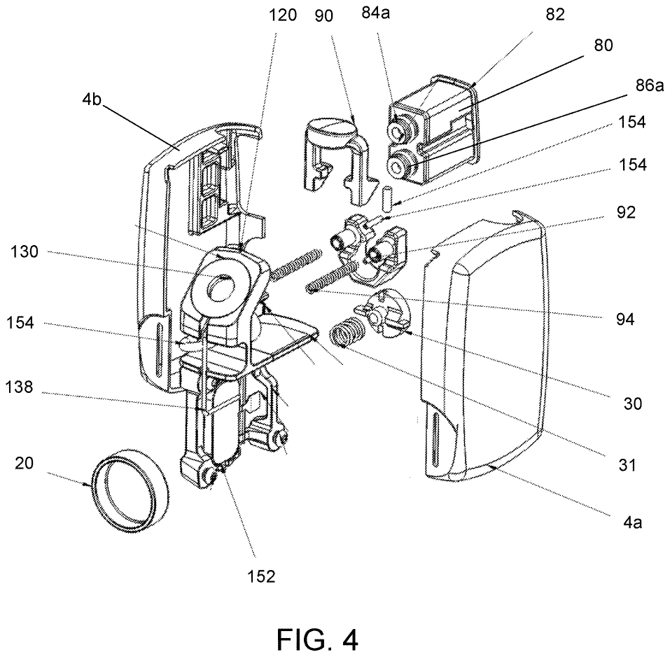

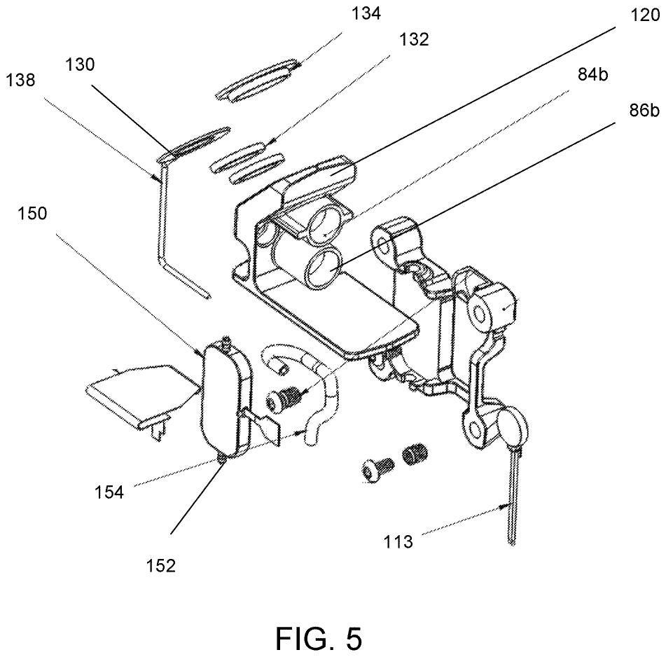

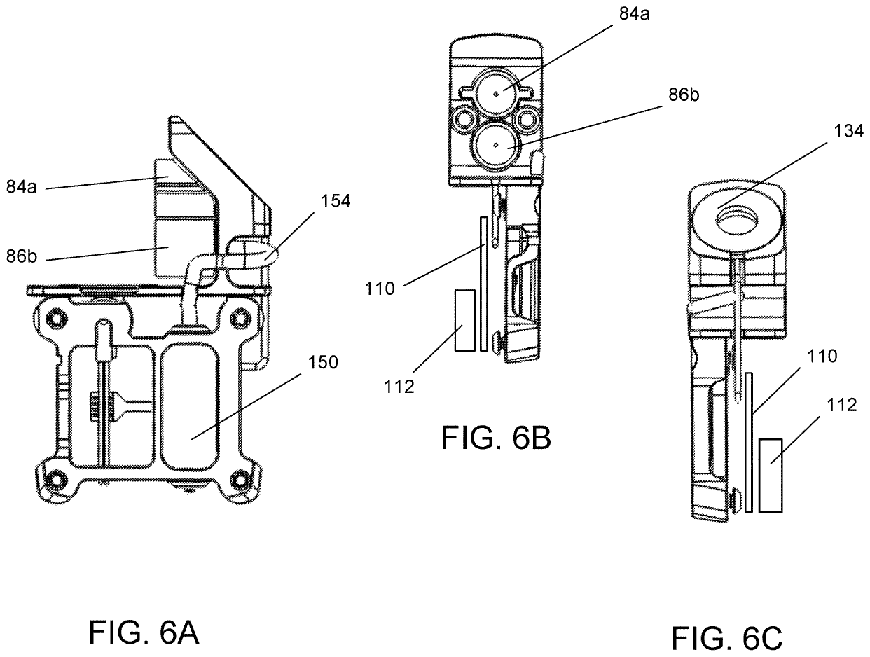

FIGS. 1 through 6C show an embodiment comprising a single main unit 2. The unit 2 comprises an outer main housing or shell 4 with one or more ports or mouthpiece units 10, 20. The housing may be unitary, or multi-part (e.g., a clamshell, with a top and bottom, or right and left sides 4a,b, where the parts may be detached from each other, or may be hingedly attached). The mouthpiece unit(s) may be detachable and replaceable. In some embodiments, the detached mouthpiece units(s) may be washable and sanitizable, and thus re-usable.

In several embodiments, the unit comprises a single exhalation port 20 into which the user breathes for some form of breath testing, as described further herein. As seen in FIGS. 1A-4, the exhalation port or mouthpiece 20 may be rotatably or slidingly attached to the housing, such as through tabs 24 that extend from the back of the port or mouthpiece 20 into slots 26 in the front of the housing, and then rotated or moved down from a home position, as seen in FIG. 1B, to a use position, as seen in FIG. 1A. The unit then performs various tests or analyses, as described below. The unit may perform a pulmonary function or breathing test (i.e., spirometry) for measuring the volume of air inspired and/or expired by the lungs of the user by pressure or ultrasonic transducers 26, effectively acting as a spirometer. The test identifies, among other things, abnormal ventilation pattern (e.g., obstructive, and restrictive). It also can be used to identify and find the cause of shortness of breath, assessing the effect of medication on lung function, and evaluating progress for treatment of certain types of lung disease or conditions (e.g., asthma, COPD, bronchitis, emphysema, and COVID-19).

In the use position, the user blows into the exhalation port or mouthpiece 20, his or her breath contacts the transducers 26 which are located on the inside of the back of the mouthpiece, and the exhaled air then passes through and exits through holes or openings 28 proximate the edges of the back. The exhaled air passageway is entirely separate from a second passageway used for the inhalation function, as described below. There is no fluid contact between the passageways. The exhaled air enters through its own port, is analyzed, and exits through its own exit ports or openings, separate from the inhalation entrance and exit ports and passageway. In a preferred embodiment, the exhaled air enters through its own port, is analyzed, and exits through its own exit ports or openings without entering the portion of the housing containing the inhalation ports and passageway. The separation prevents the possible contamination of one function by the other, and in particular, prevents elements of the exhaled breath from migrating to or contaminating the inhalation elements and function, while still providing the convenience of having a single device provide both functions.

The housing 4 further includes one or more controls, such as a button, slider, or switch 30 that may be used to turn the unit on or off, or control other functions, as described below. The button may be spring-operated 31 to switch between positions. One or more lights, LEDS, or other indicators 32 may be provided to indicate status of the unit, including, but not limited to, power status and operational status. In the embodiment shown, a LED 32 or other form of light is visible on the outer surface of the shell to indicate power status and breath volume test result. The LED or light 32 indicates when the unit is on, when the user has correctly and properly blown into the exhalation port 20 sufficiently to provide a viable test result, and a color (e.g., Green/Yellow/Red) to indicate qualitatively the results of the breath volume test. The LED or light 32 may also be used to provide similar information for the inhalation function. Other forms of audio and/or visual acknowledgment or indicators may be provided.

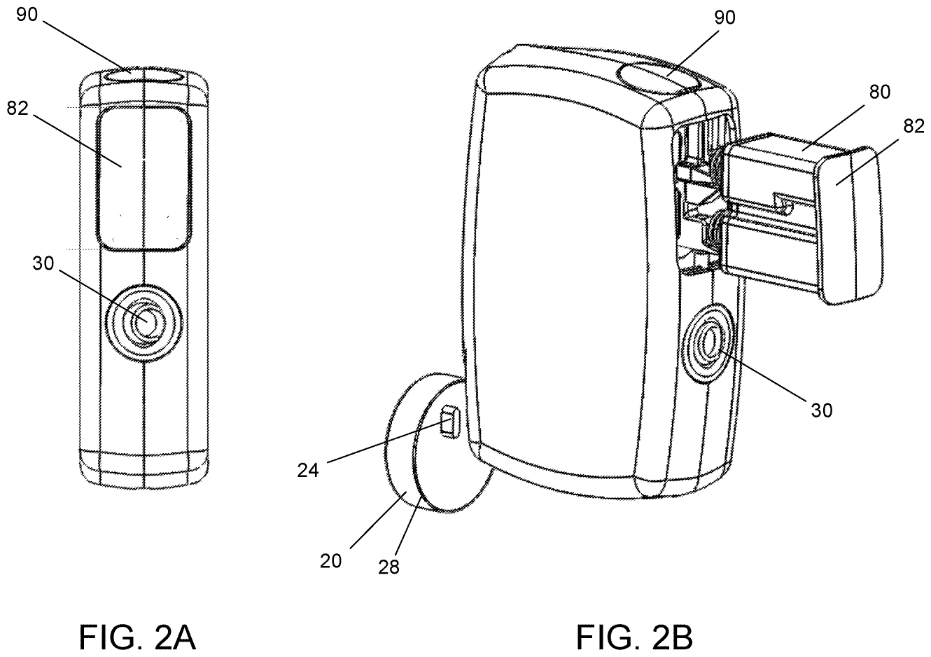

The main shell further comprises an opening for insertion of a pod, ampoule or cartridge 80 into the unit. The pod, ampoule, or cartridge 80, as seen in FIG. 2B, holds the substance to be vaporized or nebulized during operation of the unit. The vaporized substance is inhaled by the user while holding the unit. When inserted, the surface of the outer end 82 of the pod 80 acts as a cover for the opening and smoothly aligns with the contours of the housing. In some embodiments, a separate cover can be placed over the opening when a pod is not inserted therein.

As seen in the figures, in one embodiment the pod comprises an interior chamber with two ports on the back, one 84a above the other 86a. The ports extend outward from the pod and comprise an interface and a gasket configured to mate with matching ports 84b, 86b inside the unit. When inserted, the pod/ampoule/cartridge 80 is locked into place by a bracket 92 interacting with arms extending downward from the retention/release button 90. The bracket is under tension by compression of springs 94. When the retention/release button 90 is pressed down, the arms move downward, releasing the bracket 92, which is pushed backward and out from the housing by action of the springs 94. The pod thus is held securely in place, until ejected by means of the release button on the unit.

The pod/ampoule/cartridge 80 may comprise a wireless chip containing some or all of the following information: pod identification; substance/medicament identification; frequency set; intended user; user age; user weight; activate location; available doses; doses provided to the patient; a timer reminder for the user to take the substance/medicament; whether access is Face ID restricted; and confirmation of the user.

The substance to be vaporized may be in the form of a liquid, gel, gas, solid, or the like. In several embodiments, the substance comprises one or more of a therapeutic substance, homeopathic or naturopathic formulations or remedies, serums, or the like. Particular substances may be chosen or selected for particular desired effects, therapies or treatments, and substances have natural vaporization characteristics that are dependent on a variety of factors, including, but not limited to, temperature, air flow, and substance composition and chemical state. Selection of one or more substances for vaporization may be made based upon information obtained from the breath analysis tests performed by the unit 2, or from other devices or systems, such as a separate breath analysis device that can capture and analyze the content of exhaled air, a health band (e.g., Fit Bit), other detection devices, or information stored in or entered into in a health or wellness computer application, program or database on a computer or mobile device (e.g., smart phone, tablet computer), which can coordinate diagnosing this information and developing recommendations for the substance or substances to be used (as described below).

In an alternative embodiment, the unit comprises an internal, refillable chamber for holding the substance to be vaporized. The refillable chamber may be filled by insertion of the substance through the opening with removable cover, or other filling port.

In one exemplary embodiment, the interior of the unit comprises an atomization chamber, which receives the substance from the pod/ampoule/cartridge or refillable chamber through such means as a wick or other conduit. Air enters the entrance port 152 on one end of the peristaltic pump 150, and is directed through tubing 154 to the lower or inlet port(s) 86a,b associated with the pod. The substance in the pod is then transmitted out the upper or outlet port(s) 84a,b. Vaporization is achieved by means of one or more piezoelectric transducers or atomizers 130, providing sonic or ultrasonic vibration. Transducers are connected to the circuit board and power supply. Vapor is emitted through tubing or conduits to the mouthpiece support orifice 134. Two or more gaskets 132 are located between the transducer 130 and the internal mouthpiece frame 120 Calibration and control of the atomization process (e.g., temperature, size of inlet openings, rate of vaporization, timer) may be controlled by the user manually using a control on the unit, automatically controlled based upon a code or symbol (e.g., bar code) on the ampoule read by a bar code scanner in the device after insertion, or remotely (such as by wireless connection to a computer or mobile device). This calibration and control may be performed according to the diagnosis and recommendations developed in the manner described above.

A circuit board 110 provides control and power functions. Power may be provided by one or more batteries 112. The battery or batteries may be standard, replaceable batteries, or may be a rechargeable battery built into the unit, and recharged with a recharging cord or similar means. In some embodiments, a power cord and plug may be plugged into a standard electrical outlet to provide power. A wifi, Near Field Communication (NFC) and/or Bluetooth chip provides for wireless communications, in conjunction with antenna 113. A USB, mini-USB, or similar communications and/or data port 40 provides for direct communications, and uploading and downloading of programs or data. The USB port or other port 40 may also be used as a power source, and may be used to recharge the rechargeable battery 112. In several embodiments, the unit may use wireless charging or other form of self-inducting charging.

Other vaporization means may be provided, such as a heat source (burner, flame, electrical). Temperature may be controlled in the manner of other parameters discussed above. In several embodiments, the temperature is regulated to keep the substance cool to enhance the stability of the chemical constituents thereof. In yet another embodiment, a unit has multiple vaporization elements, and can vaporize different substances from one or more, or multiple, refillable chambers or multiple ampoules simultaneously, in sequence, or some combination thereof.

The unit may have a computer memory storage capability, and store vaporization treatment data so that the details of the vaporization treatment can be subsequently used to evaluate clinical or medical treatment compliance and effectiveness. The information may be provided by wired or wireless connection to a health or wellness program. The unit may have one or more means of wireless communication and wired/or communication (e.g., data ports, USB ports, telecommunications ports). The unit may transmit information and data to (and received the same from) one or more remote servers and databases, and may be “cloud-based.” As mentioned above, information and data may also be provided to a physicians'or health-care providers'portal (and device), who may receive alerts about users/patients who have had unusual metrics (e.g., a indicator exceeding a pre-set threshold), and thus be able to review the data and take action, as appropriate.

The atomization chamber and conduit may be cleaned between uses using a cleaning solution, by one or more interior UV light or radiation sources along the chamber and conduit, or combinations thereof.

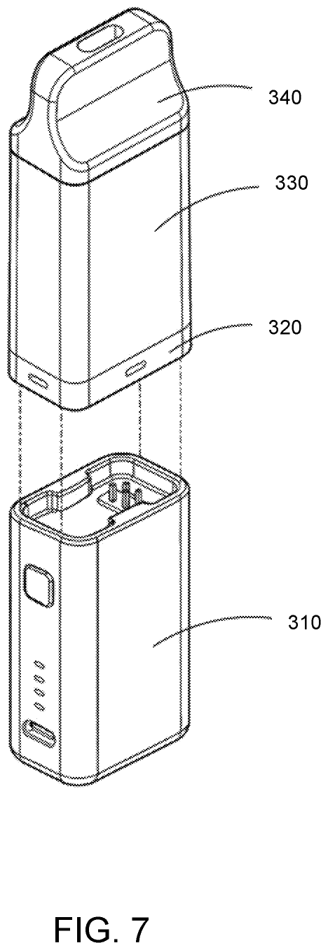

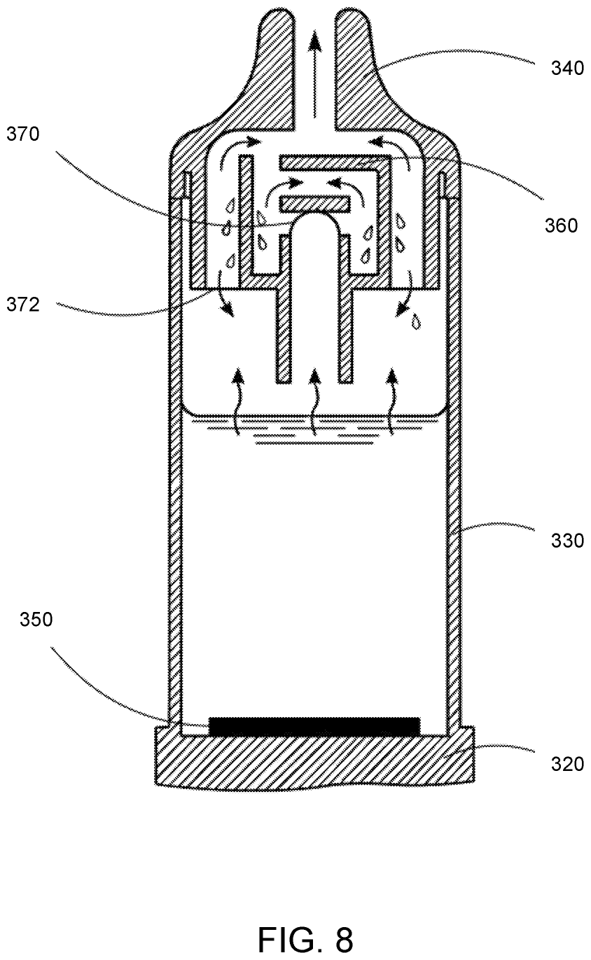

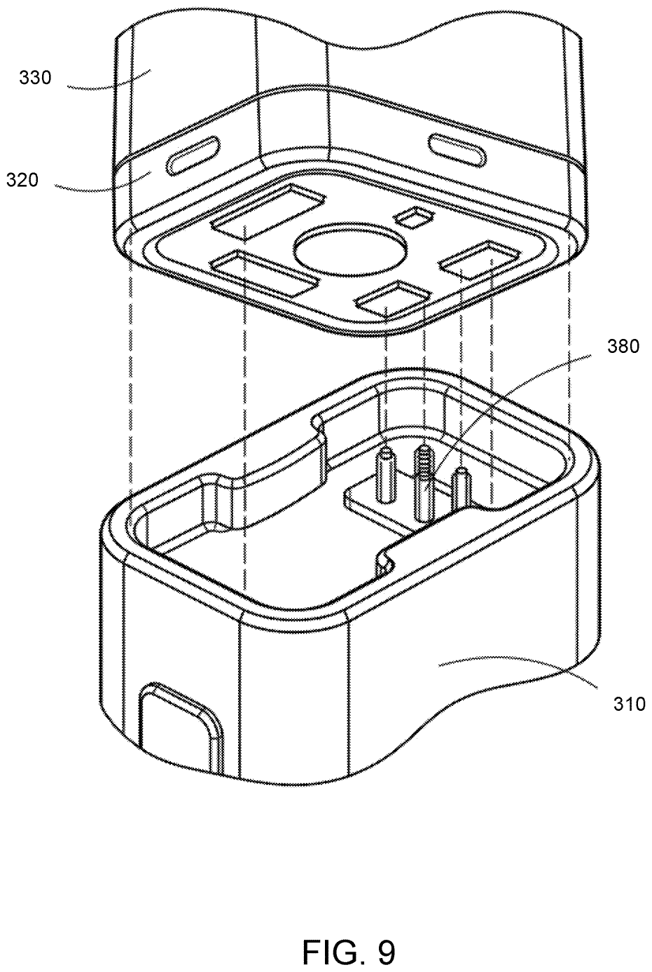

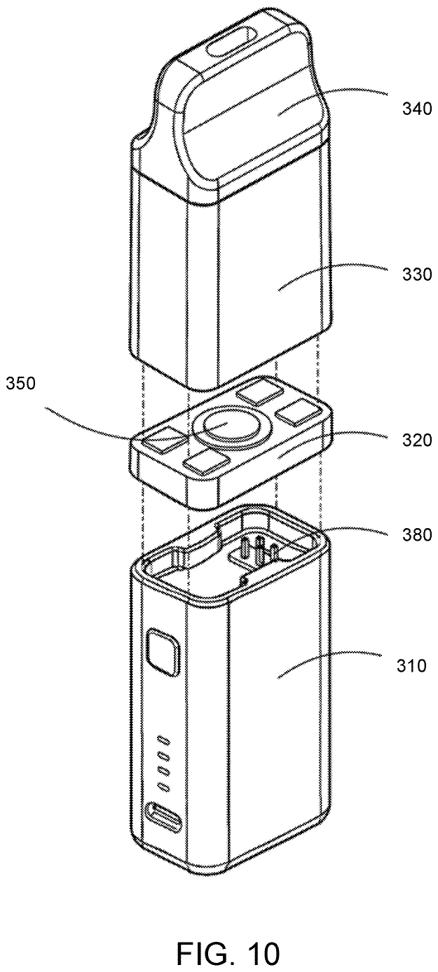

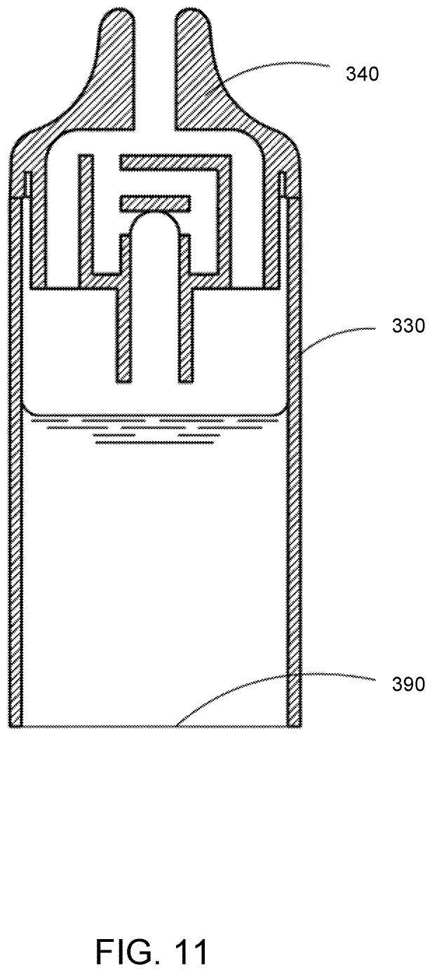

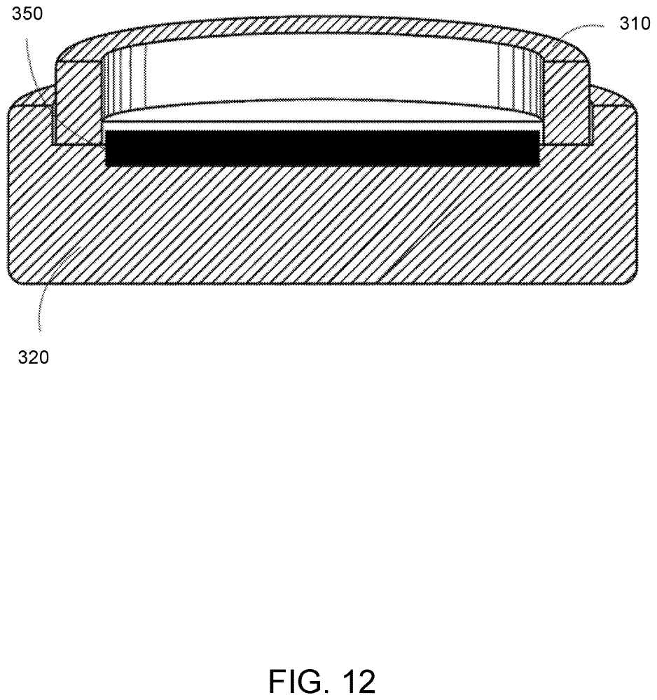

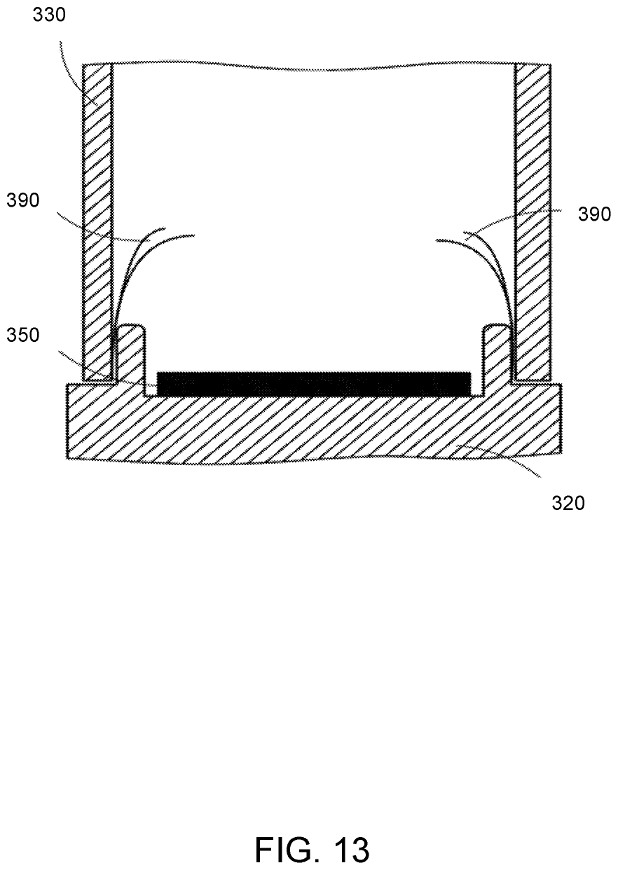

FIGS. 7-13 show examples of modular systems for an inhaler system. FIGS. 7-9 show an example of a two component (i.e., two tier) system, while FIGS. 10-13 show a three component (i.e., three tier) system.

As seen in FIGS. 7-9, the cartridge system of the two-tier embodiment comprises a cartridge and mouthpiece unit, which comprises a sealed housing 330 defining an interior liquid reservoir 332. The housing 330 is formed of materials suitable for containing pharmaceutical or therapeutic liquids, including but not limited to medical-grade plastics, glass, or combinations thereof. A piezoelectric transducer 350 is positioned at the bottom interior surface of the housing 330. The piezoelectric transducer 350 comprises a piezoelectric disc, which may be formed of lead zirconate titanate (PZT), barium titanate, or other suitable piezoelectric materials. The piezoelectric transducer 350 is oriented such that liquid within the liquid reservoir 332 pools on the upper surface of the piezoelectric transducer 350 via gravity, ensuring consistent contact between the liquid and the nebulization surface.

At the top of the cartridge/upper unit, a mouthpiece assembly 340 provides the user interface for inhalation. The mouthpiece assembly 340 includes an air intake pathway 374, a vapor delivery pathway, and vapor/liquid separation mechanisms 370. The vapor/liquid separation mechanisms 370 may comprise one or more of: a labyrinth/baffle system 360 that prevents liquid droplets from reaching the user, a mesh or screen that allows vapor passage while blocking liquid, a condensation surface that captures and returns liquid to the liquid reservoir 332, or a labyrinthine pathway 360 that separates liquid from vapor through momentum-based separation. The mouthpiece assembly 340 may be integrally formed with the cartridge/upper unit housing or may be a separate component that attaches to the housing.

The bottom exterior surface of the cartridge unit includes electrical contacts 380 for connection to the base device or unit 310. The electrical contacts 380 provide electrical pathways for delivering power to the piezoelectric transducer 350 and, in certain embodiments, for bidirectional data communication between the cartridge and base device 310.

The cartridge includes one or more identification components that enable the base device 310 and associated systems to identify, authenticate, and track the cartridge. These identification components may include, individually or in combination:
(a) Wired identification through the electrical contacts 380, wherein identification data is transmitted via the same physical connection used for power delivery. This may include an EEPROM or similar non-volatile memory storing identification data, a resistor or resistor network providing a resistance value that encodes cartridge information, or a simple circuit that provides a predetermined electrical signature.
(b) Wireless identification components including an NFC (Near Field Communication) chip and antenna, an RFID (Radio Frequency Identification) tag operating at LF, HF, or UHF frequencies, or a Bluetooth Low Energy beacon.
(c) Optical identification including a QR code, barcode, or other machine-readable optical pattern printed or etched on the cartridge exterior.

The identification data may include cartridge serial number, manufacturing date, expiration date, liquid formulation type and concentration, batch or lot number, maximum recommended usage, and authentication codes for anti-counterfeiting purposes.

The base device 310 comprises a housing containing a rechargeable battery, electronic control circuitry, and a cartridge interface.

The rechargeable battery may be a lithium-ion, lithium-polymer, or other suitable rechargeable cell. The battery provides power for the piezoelectric transducer 350 in the cartridge, the electronic control circuitry, and wireless communication. A charging interface, which may comprise a USB-C port, micro-USB port, wireless charging coil, or proprietary connector, enables recharging of the battery.

The electronic control circuitry includes a microcontroller or processor, memory for storing firmware and usage data, a driver circuit for generating the high-frequency signal required by the piezoelectric transducer 350, and a wireless communication module. The wireless communication module may include Bluetooth Low Energy (BLE) capability for communication with a mobile device application, and may additionally include WiFi, cellular, or other wireless protocols.

The cartridge interface includes an attachment mechanism for mechanically securing the cartridge unit 330 to the base device 310. The attachment mechanism may comprise threading, a bayonet mount, a magnetic connection, a snap-fit engagement, a friction fit, or combinations thereof. The interface further includes electrical contacts 380 corresponding to those on the cartridge, positioned to make electrical connection when the cartridge is properly attached. In embodiments utilizing wireless identification, the base device 310 includes an appropriate reader, such as an NFC reader or RFID reader, positioned to read the identification component on the cartridge when attached or during the attachment process.

The cartridge/mouthpiece unit of the three-tier embodiment is similar in general form to the cartridge/mouthpiece of the two-tier embodiment described above. Unlike the two-tier embodiment, however, the cartridge unit 330 of the three-tier embodiment does not include an integrated piezoelectric transducer 350, but instead interacts with a removable/replaceable piezoelectric nebulization module 320. The nebulization module 330 is disposed between the cartridge/mouthpiece unit 330 and the base device 310. This modular arrangement reduces the cost of incorporating a transducer 350 in every cartridge unit 330, and allows easier repair and/or replacement of the transducer 350, if needed. In a similar fashion, the piezoelectric nebulization transducer 350 and associated components can be placed in the base device 310, although this arrangement does not have the advantage allowing easier repair and/or replacement of the transducer and associated components by replacement of a separable module 320.

As described above, the base device 310 includes one or more user interface elements, which may include a button for activating nebulization, LED indicators for status indication, a display, haptic feedback elements, or combinations thereof. A button for activating nebulization has several advantages over activation by detecting user inhalation, including, but not limited to, reducing cost and avoiding a point of failure with an airflow and/or pressure sensor in an extended airflow passageway.

In the three-tier embodiment, the bottom surface of the cartridge unit 330 includes a sealed membrane 390 that maintains the liquid contents in a sealed state prior to use. When the cartridge unit is engaged with the piezoelectric nebulization module 320, the seal membrane 390 is broken to allow the liquid access to the nebulization surface below.

The cartridge unit includes identification components as described for the two-tier embodiment. Alternatively, the primary identification may be through wireless means (e.g., NFC or RFID) as the electrical contacts 380 between the cartridge unit 330 and piezoelectric nebulization module 320 may be minimal or absent. However, wired identification through the piezoelectric module 320 to the base device 310 remains an option as well.

The piezoelectric nebulization module 320 is a distinct component positioned between the cartridge unit 330 and the base device 310. The module comprises a piezoelectric module housing containing a piezoelectric transducer 350, electrical contacts 380 on both upper and lower surfaces, and further may include module-specific identification components.

The upper surface of the piezoelectric module comprises an interface for engaging with the cartridge unit. This interface includes a seal-breaking mechanism 324 that, upon engagement of the cartridge, penetrates or ruptures the seal membrane 390 on the cartridge bottom to allow liquid to flow onto the piezoelectric transducer 5 surface. The seal-breaking mechanism 324 may comprise a puncture element such as a spike, needle, or blade that actively penetrates the seal membrane 390, a shaped surface that causes the seal membrane 390 to rupture under pressure when the cartridge is pressed into engagement, or a combination of active and passive seal-breaking elements.

The attachment mechanism between the cartridge unit 330 and the piezoelectric module 320 may comprise threading, a bayonet mount, a magnetic connection, a snap-fit engagement, or combinations thereof.

The lower surface of the piezo module housing 320 includes electrical contacts 380 for connection to the base device 310. The electrical contacts 380 provide pathways for power delivery to the piezoelectric transducer 5 and for data communication. In certain embodiments, the module includes an EEPROM or similar memory that stores module identification data and may additionally store usage data such as cumulative operating time, number of nebulization cycles, or date of first use.

The piezoelectric module is designed for periodic replacement, with an expected service life of approximately two to three months under typical use conditions. The module identification components enable the base device 310 to track module usage and alert the user when replacement is recommended based on cumulative use, elapsed time since first use, or detected performance degradation.

The base device 310 of the three-tier embodiment is substantially similar to that of the two-tier embodiment, comprising a rechargeable battery, electronic control circuitry, wireless communication capability, and user interface elements. The module interface on the base device 310 includes an attachment mechanism and electrical contacts 380 corresponding to those on the piezoelectric nebulization module housing 320. In embodiments where the cartridge includes wireless identification components, the base device 310 additionally includes an appropriate wireless reader (NFC or RFID) positioned to read the cartridge identification through the piezoelectric module or when the cartridge is in proximity to the base device 310.

The electronic control circuitry is configured to communicate with both the piezoelectric module and the cartridge, tracking usage of each component independently. The circuitry generates appropriate notifications through the user interface elements and/or through a connected mobile application when the piezoelectric module requires replacement or when cartridge-specific conditions (such as low liquid or expiration) are detected.

Various embodiments of the present invention, including those described above, are designed for integration with a mobile application running on a smartphone, tablet, or other computing device. The mobile application communicates with the base device 310 via Bluetooth Low Energy or other wireless protocol. The mobile application may provide functionality including display of current cartridge information (formulation, expiration, remaining usage), historical usage tracking and visualization, reminder notifications for cartridge replacement or medication schedules, authentication verification of genuine consumables, integration with health records or care provider systems, and device settings and firmware updates.

In the three-tier embodiment, the mobile application additionally provides tracking of piezoelectric module status, including cumulative use time, estimated remaining service life, and replacement reminders.

Activation of nebulization may be initiated by pressing a button on the base device 310, receiving a voice, electronic, or other form of command from the mobile application, or combinations of these activation methods.

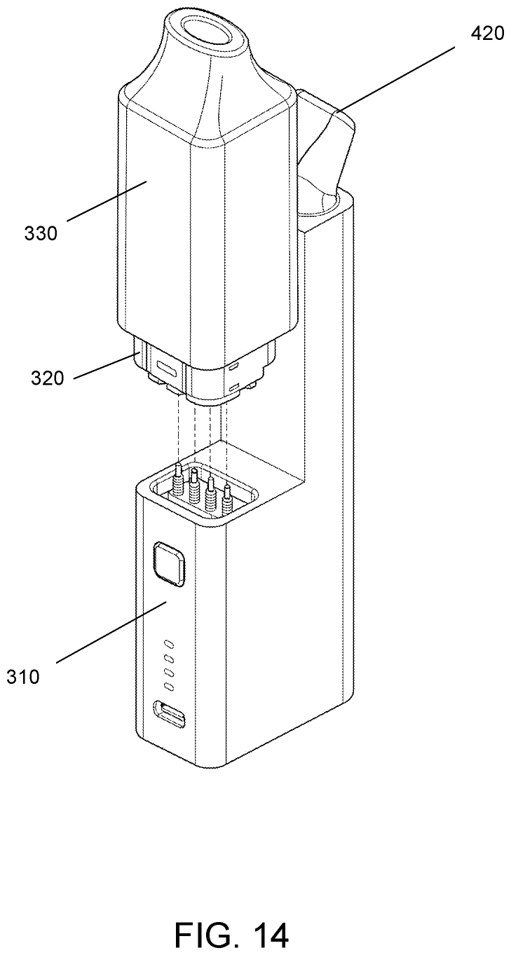

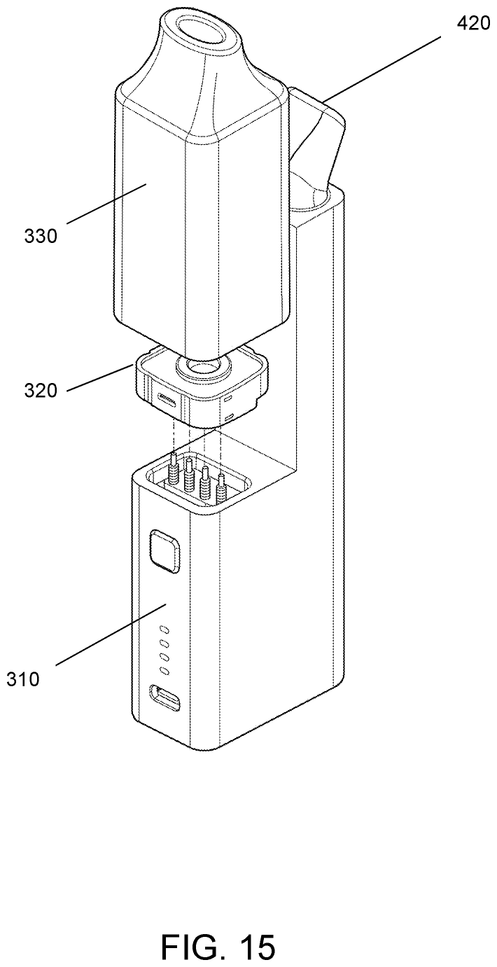

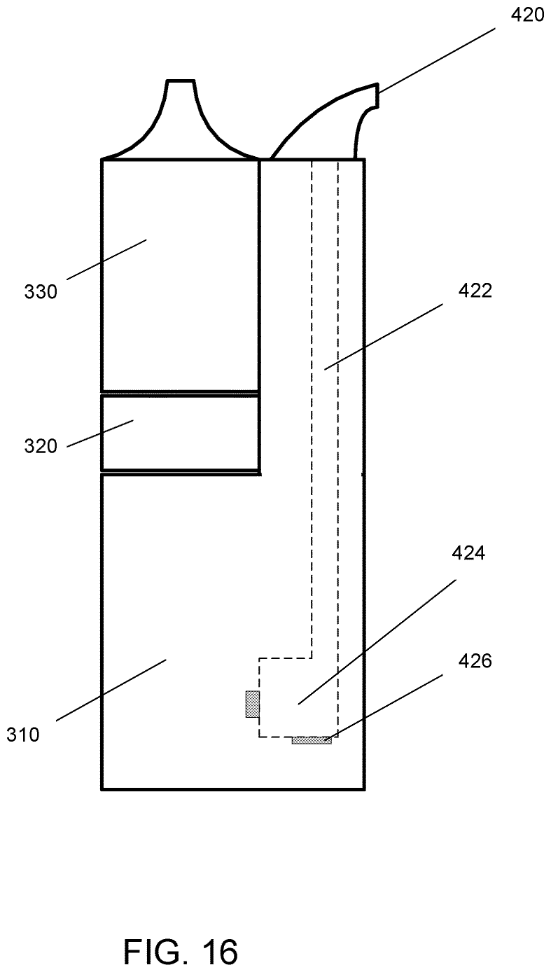

FIGS. 14-18 show examples of modular systems that combine both inhalation and exhalation functions, in a similar manner as that described above for the embodiments shown in FIG. 1-6C. FIGS. 14-16 show a three-tier modular system where the base unit 310 has been modified to provide a path for a user to breath into (i.e., exhale) a mouthpiece or exhale port 420 positioned on the top of the base unit; exhaled air travel in a separate passageway 422 inside the base unit from the mouthpiece 420 to an analysis chamber or component 424 where analysis of the breath is performed by one or more transducers or sensors 426. The piezoelectric module 320 and the cartridge/mouthpiece unit 330 for the inhalation function are attached to a lower section of the upper surface of the base unit, and as described above, the passageway 422 and mouthpiece for the exhalation analysis function is completely separate and apart from any passageways and mouthpieces for the inhalant function. Sensors may include, but are not limited to, capnography devices, temperature sensors, water vapor sensors, blood alcohol sensors, airflow sensor, pressure sensors and pH sensors. Sensors also may be used to detect the presence or concentration of one or more of the therapeutic substances provided by the personal nebulizer or vaporizing unit.

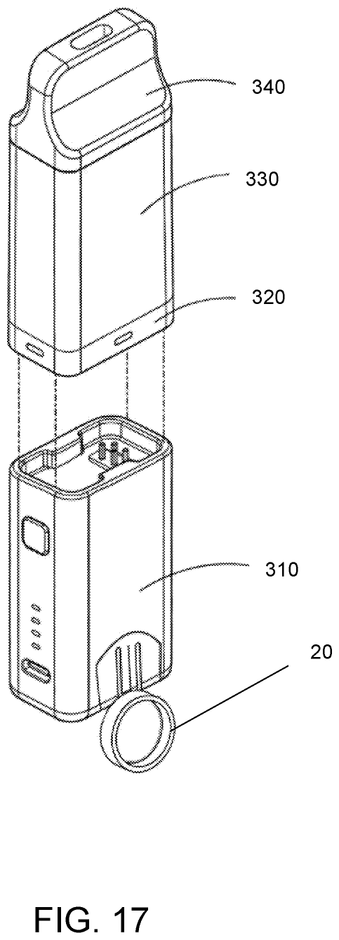

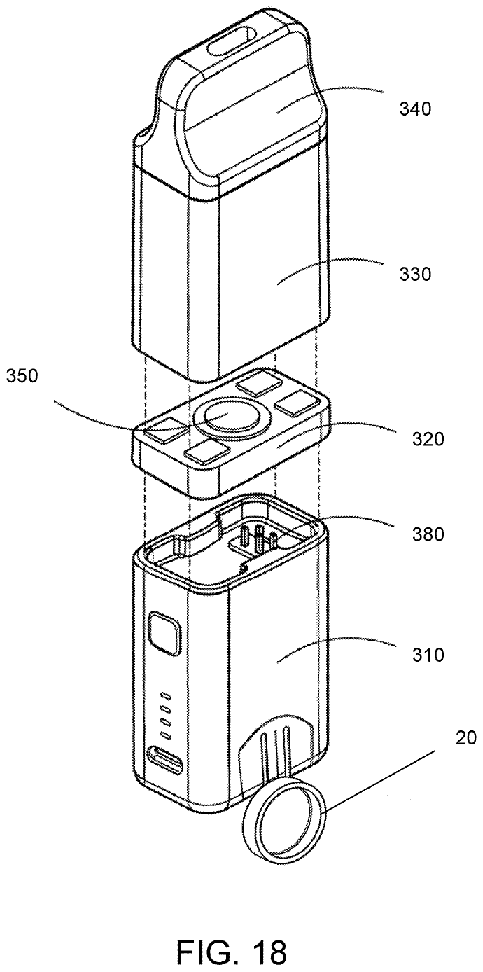

FIGS. 17-18 shows an alternative embodiment of a dual-port system, wherein the exhalation port 20 is located on the exterior of base unit 310 in a similar manner, and operates in a similar manner, to that described above for the embodiments shown in FIG. 1-6C.

The same base unit dual-port modifications described above for the three-tier modular systems can also be used for the two-tier modular systems

While the above embodiments have been described for management and treatment of asthma, the present invention also may be used for management and treatment of one or more other conditions, including, but not limited to, COPD, diabetes, cystic fibrosis, and other forms of acute and chronic diseases. The present invention may also be used to provide palliative treatments, such as pain-killers, cannabinoids (e.g., CBD), and the like, especially those taken on a schedule.

In order to provide context for the various computer-implemented aspects of the invention, the following discussion provides a brief, general description of a suitable computing environment in which the various aspects of the present invention may be implemented. A computing system environment is one example of a suitable computing environment, but is not intended to suggest any limitation as to the scope of use or functionality of the invention. A computing environment may contain any one or combination of components discussed below, and may contain additional components, or some of the illustrated components may be absent. Various embodiments of the invention are operational with numerous general purpose or special purpose computing systems, environments or configurations. Examples of computing systems, environments, or configurations that may be suitable for use with various embodiments of the invention include, but are not limited to, personal computers, laptop computers, computer servers, computer notebooks, hand-held devices, mobile computing devices, microprocessor-based systems, multiprocessor systems, TV set-top boxes and devices, programmable consumer electronics, cell phones, personal digital assistants (PDAs), tablets, smart phones, mobile phones, modular phones, touch screen devices, smart TV, TVs, internet-enabled appliances, internet-enabled security systems, internet-enabled gaming systems, internet-enabled watches; internet-enabled cars (or transportation), network PCs, minicomputers, mainframe computers, embedded systems, virtual systems, distributed computing environments, streaming environments, volatile environments, and the like.

Embodiments of the invention may be implemented in the form of computer-executable instructions, such as program code or program modules, being executed by a computer, virtual computer, or computing device. Program code or modules may include programs, objects, components, data elements and structures, routines, subroutines, functions and the like. These are used to perform or implement particular tasks or functions. Embodiments of the invention also may be implemented in distributed computing environments. In such environments, tasks are performed by remote processing devices (e.g., “cloud computing”) linked via a communications network or other data transmission medium, and data and program code or modules may be located in both local and remote computer storage media including memory storage devices such as, but not limited to, hard drives, solid state drives (SSD), flash drives, USB drives, optical drives, and internet-based storage (e.g., “cloud” storage).

In one embodiment, a computer system or computing system environment comprises multiple client devices in communication with one or more server devices through or over a network, although in some cases no server device is used. In various embodiments, the network may comprise the Internet, an intranet, Wide Area Network (WAN), or Local Area Network (LAN). It should be noted that many of the methods of the present invention are operable within a single computing device.

A client device may be any type of processor-based platform that is connected to a network and that interacts with one or more application programs. A client device may comprise a computer-readable medium in the form of volatile and/or nonvolatile memory such as read only memory (ROM) and random access memory (RAM) in communication with a processor. The processor executes computer-executable program instructions stored in memory. Examples of such processors include, but are not limited to, microprocessors, ASICs, and the like.

Client devices may further comprise computer-readable media in communication with the processor, said media storing program code, modules and instructions that, when executed by the processor, cause the processor to execute the program and perform the steps described herein. Computer readable media can be any available media that can be accessed by computer or computing device and includes both volatile and nonvolatile media, and removable and non-removable media. Computer-readable media may further comprise computer storage media and communication media. Computer storage media comprises media for storage of information, such as computer readable instructions, data, data structures, or program code or modules. Examples of computer-readable media include, but are not limited to, any electronic, optical, magnetic, or other storage or transmission device, a floppy disk, hard disk drive, CD-ROM, DVD, magnetic disk, memory chip, ROM, RAM, EEPROM, flash memory or other memory technology, an ASIC, a configured processor, CD-ROM, DVD or other optical disk storage, magnetic cassettes, magnetic tape, magnetic disk storage or other magnetic storage devices, or any other medium from which a computer processor can read instructions or that can store desired information. Communication media comprises media that may transmit or carry instructions to a computer, including, but not limited to, a router, private or public network, wired network, direct wired connection, wireless network, other wireless media (such as acoustic, RF, infrared, or the like) or other transmission device or channel. This may include computer readable instructions, data structures, program modules or other data in a modulated data signal such as a carrier wave or other transport mechanism. Said transmission may be wired, wireless, or both. Combinations of any of the above should also be included within the scope of computer readable media. The instructions may comprise code from any computer-programming language, including, for example, C, C++, C #, Visual Basic, Java, and the like.

Components of a general purpose client or computing device may further include a system bus that connects various system components, including the memory and processor. A system bus may be any of several types of bus structures, including, but not limited to, a memory bus or memory controller, a peripheral bus, and a local bus using any of a variety of bus architectures. Such architectures include, but are not limited to, Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computing and client devices also may include a basic input/output system (BIOS), which contains the basic routines that help to transfer information between elements within a computer, such as during start-up. BIOS typically is stored in ROM. In contrast, RAM typically contains data or program code or modules that are accessible to or presently being operated on by processor, such as, but not limited to, the operating system, application program, and data.

Client devices also may comprise a variety of other internal or external components, such as a monitor or display, a keyboard, a mouse, a trackball, a pointing device, touch pad, microphone, joystick, satellite dish, scanner, a disk drive, a CD-ROM or DVD drive, or other input or output devices. These and other devices are typically connected to the processor through a user input interface coupled to the system bus, but may be connected by other interface and bus structures, such as a parallel port, serial port, game port or a universal serial bus (USB). A monitor or other type of display device is typically connected to the system bus via a video interface. In addition to the monitor, client devices may also include other peripheral output devices such as speakers and printer, which may be connected through an output peripheral interface.

Client devices may operate on any operating system capable of supporting an application of the type disclosed herein. Client devices also may support a browser or browser-enabled application. Examples of client devices include, but are not limited to, personal computers, laptop computers, mobile computing devices, personal digital assistants, computer notebooks, hand-held devices, cellular phones, mobile phones, modular phones, smart phones, pagers, digital tablets, Internet appliances, and other processor-based devices. Users may communicate with each other, and with other systems, networks, and devices, over the network through the respective client devices.

Thus, it should be understood that the embodiments and examples described herein have been chosen and described in order to best illustrate the principles of the invention and its practical applications to thereby enable one of ordinary skill in the art to best utilize the invention in various embodiments and with various modifications as are suited for particular uses contemplated. Even though specific embodiments of this invention have been described, they are not to be taken as exhaustive. There are several variations that will be apparent to those skilled in the art.

What is claimed is:

1. A combined testing and treatment device comprising:
a first unit comprising
a housing with an exterior and an interior;
a reservoir in the interior, said reservoir containing a substance;
a piezoelectric transducer configured to nebulize some of all of the substance;
an inhalation mouthpiece or mouth port in or on the housing, configured to
emit the nebulized substance to a user; and
a first electronic contact interface on the exterior of the housing;
a base device comprising
a base housing with an interior and an exterior;
a battery in the interior of the base housing;
a second electronic contact interface on the exterior of the base housing, wherein the first electronic contact interface and the second electronic contact interface are configured to removably connect to each other; and
an exhalation mouthpiece or mouth port in or on the base housing, configured to receive and direct exhaled breath of the user to an analysis transducer configured to detect one or more properties of the exhaled breath;
wherein the inhalation mouth port or mouthpiece is separate from the exhalation mouth port or mouthpiece, and the inhalation mouthpiece or mouth port and the exhalation mouthpiece or mouth port do not share any portion of a fluid passageway; and
wherein the exhaled breath does not come into contact with the first piezoelectric transducer.

2. The device of claim 1, wherein the device provides both an inhalation function with one or more first fluid passageways via the inhalation mouthpiece or mouth port, and an exhalation function with a second fluid passageway via the exhalation mouthpiece or mouth port, wherein the one or more first fluid passageways are separate from the second fluid passageway.

3. The device of claim 1, wherein the exhaled breath does not enter the interior of the base housing.

4. The device of claim 1, wherein the second transducer measures pressure.

5. The device of claim 4, wherein the device with exhalation port and second transducer is configured to act as a spirometer.

6. The device of claim 1, wherein the exhalation port is slidingly or rotatably mounted on the base housing.

7. The device of claim 1, further comprising at least one wireless communication chip located in the interior of the first unit housing or the interior of the base unit housing.

8. The device of claim 1, wherein the device is portable and hand-held.

9. The device of claim 1, wherein the exhaled breath enters the interior of the base housing.

10. The device of claim 1, wherein the second transducer measures one or more of temperature, water vapor content, humidity, blood alcohol, airflow volume, and pH.

11. The device of claim 7, wherein the device is in wireless communication with a mobile computing device with a microprocessor and a mobile device wireless communication chip, wherein the microprocessor is programmed to:
store data about the user, said data comprising identification data and health data;
receive, in real time, via electronic communications from the device, information about the substance in the first unit;
receive, in real times, via electronic communications from the device, information about the user's use of the substance contained in the inserted ampoule.
